(19) United States
(12) Patent Application Publication
(10) Pub. No.: US 2019/0142361 A1
(43) Pub. Date: May 16, 2019

(54) DIAPHRAGM SET FOR STETHOSCOPE AND DISPOSABLE CAPSULE HAVING THE SAME

(71) Applicant: VITALCHAINS CORPORATION, Menlo Park, CA (US)

(72) Inventors: KUN HSI TSAI, Chupei (TW); TZU CHIH LIN, Chupei (TW); BING WEI WENG, Chupei (TW); YI TING HUANG, Chupei (TW); YU CHENG HUANG, Chupei (TW)

(21) Appl. No.: 16/181,352

(22) Filed: Nov. 6, 2018

Related U.S. Application Data
(60) Provisional application No. 62/584,215, filed on Nov. 10, 2017.

(51) Int. Cl. A61B 7/02; G10K 13/00

(52) U.S. Cl. CPC A61B 7/02; A61B 2562/242; G10K 13/00

(57) ABSTRACT

The present disclosure provides a diaphragm set for a stethoscope having a chest piece. The diaphragm set includes a diaphragm having a bottom structure and a side structure connected to an edge of the bottom structure. The bottom structure and the side structure of the diaphragm form a diaphragm cavity for receiving the chest piece of the stethoscope.

### CROSS-REFERENCE TO RELATED APPLICATION

This application claims priority to U.S. Provisional Patent Application No. 62/584,215, filed on Nov. 10, 2017, the entirety of which is incorporated herein by reference.

### FIELD

The present disclosure generally relates to a disposable diaphragm for a stethoscope. More particularly, the present disclosure relates to a diaphragm set for a stethoscope and a disposable capsule containing the diaphragm set.

### BACKGROUND

A stethoscope is a medical instrument that allows a doctor to listen to a patient's internal body sounds, such as heartbeats, by placing the head of the stethoscope onto the patient's skin. As shown in FIG. 1A, the stethoscope 10 includes a stethoscope head 100 and a stethoscope tube 200 connected to the stethoscope head 100, and two earpieces 210. The stethoscope tube 200 has one proximal end and two distal ends. As shown in FIGS. 1B to 1D, the stethoscope head usually includes a chest piece 110, a diaphragm 120 and an O-ring 130. The proximal end of the stethoscope tube 200 is connected to the chest piece 110. The two distal ends of the stethoscope tube 200 are respectively connected to the two earpieces 210. The diaphragm 120 covers an opening of the chest piece 110. The diaphragm 120 is bound to the chest piece 110 by the O-ring 130 to form an airtight sealing with the chest piece 110. The vibration produced by the patient's internal body sounds is transmitted through the diaphragm 120 to the chest piece and then the stethoscope tube 200, so that the doctor can hear the internal body sounds through the earpieces 210 connected to two the distal ends of the stethoscope tube 200.

In order to prevent the transmission of infections between patients, the diaphragm 120 must be replaced after use. However, during the replacement process, mismatching of the diaphragm 120 and the chest piece 110 may cause bad auscultation experience due to the poor sealing. Moreover, when the O-ring 130 is too small or has no flexibility to be removed or installed, the diaphragm 120 may fold or break. An alternative way to prevent the transmission of infections is to disinfect used diaphragms by alcohol. However, alcohol might affect the sensibility of the diaphragm; therefore, frequent replacement of the diaphragm is still required due to the fragility of the diaphragm's structure.

Therefore, there is a need to provide a more efficient way to replace the diaphragm of the stethoscope.

### SUMMARY OF THE INVENTION

In view of above, an object of the present disclosure is to provide a disposable diaphragm set and a disposable capsule having the diaphragm set.

To achieve the above object, an embodiment of the present disclosure provides a diaphragm set for a stethoscope having a chest piece. The diaphragm set includes a diaphragm having a bottom structure and a side structure connected to an edge of the bottom structure. The bottom structure and the side structure of the diaphragm form a diaphragm cavity for receiving the chest piece of the stethoscope.

To achieve the above object, another embodiment of the present disclosure provides a disposable capsule. The disposable capsule includes a case and a diaphragm set for a stethoscope having a chest piece. The case includes a holder having a capsule cavity and a sealing film sealing the capsule cavity of the holder. The diaphragm set is disposed in the capsule cavity of the case.

To achieve the above object, yet another embodiment of the present disclosure provides a capsule dispenser. The capsule dispenser includes a dispenser housing and at least one disposable capsule disposed in the dispenser housing. The disposable capsule includes a diaphragm set and a case for receiving the diaphragm set. The diaphragm set includes a diaphragm having a bottom structure and a side structure connected to an edge of the bottom structure. The bottom structure and the side structure of the diaphragm form a diaphragm cavity for receiving the chest piece of the stethoscope. The case includes a holder and a sealing film. The holder has a capsule cavity for receiving the diaphragm set. The sealing film seals the capsule cavity of the holder. The dispenser housing has a peelable portion. The peelable portion is configured to be peeled off from the dispenser housing to form a dispensing hole on the dispenser housing to allow the disposable capsule to be removed from the dispenser housing.

In a preferred embodiment, the side structure of the diaphragm is curved inwardly with respect to a center line of the diaphragm. The side structure includes a connecting portion connected to the bottom structure of the diaphragm, a middle portion connected to the connecting portion, and an upper portion connected to the middle portion. A radius of curvature of the middle portion is smaller than radius of curvatures of the connecting portion and the upper portion. The diaphragm set further includes an O-ring disposed around an outer surface of the side structure of the diaphragm.

In another preferred embodiment, the side structure of the diaphragm is curved outwardly with respect to a center line of the diaphragm. The side structure includes a connecting portion connected to the bottom structure of the diaphragm, a middle portion connected to the connecting portion, and an upper portion connected to the middle portion. A radius of curvature of the middle portion is greater than radius of curvatures of the connecting portion and the upper portion.

In another preferred embodiment, the diaphragm set includes a diaphragm and a rim element. The rim element couples to an edge of the diaphragm to form a cavity for receiving the chest piece of the stethoscope.

As described above, the diaphragm set of the embodiments of the present disclosure allows a user to assemble the diaphragm set to a chest piece of a stethoscope without damaging the diaphragm. In addition, the disposable capsule containing the diaphragm set of the embodiments of the present disclosure allows the user to easily replace the diaphragm set of the stethoscope without touching the diaphragm set, therefore preventing contamination and damage of the diaphragm. Furthermore, the capsule dispenser allows the disposable capsules to be neatly stored and easily retrieved.

### BRIEF DESCRIPTION OF THE DRAWINGS

Implementations of the present technology will now be described, by way of example only, with reference to the attached figures.

FIG. 1A is a perspective view of a stethoscope according to the prior art; FIG. 1B is a cross-sectional view of a stethoscope head of FIG. 1A; FIGS. 1C and 1D are a top view and a bottom of the stethoscope head of FIG. 1A.

FIG. 2A is a perspective view of a disposable capsule for a diaphragm set according to an embodiment of the present disclosure; FIG. 2B is a schematic illustration showing a sealing film of the disposable capsule of FIG. 2A being peeled off from a holder of the disposable capsule; FIG. 2C is a schematic illustration showing a diaphragm set of the disposable capsule of FIG. 2A being assembled to a chest piece of a stethoscope.

FIG. 3A is a perspective view of a case of the disposable capsule of FIG. 2A; FIG. 3B is an exploded view of the case of FIG. 3A.

FIGS. 4A and 4B are a top view and a side view of a holder of the case in FIG. 3A.

FIG. 5A is a perspective view of a diaphragm set of the disposable capsule of FIG. 2A; FIG. 5B is a perspective view from another viewing angle of the diaphragm set of FIG. 5A; FIGS. 5C and 5D are a top view and a bottom view of the diaphragm set of FIG. 5A; FIG. 5E is a side view of the diaphragm set of FIG. 5A; FIG. 5F is a cross-sectional view of the diaphragm set of FIG. 5C along plane A-A′; FIGS. 5G and 5H are partially enlarged views of FIG. 5F.

FIG. 6A is a perspective view of a diaphragm set according to another embodiment of the present disclosure; FIG. 6B is a cross-sectional view of the diaphragm set of FIG. 6A along plane B-B′; FIG. 6C is a partially enlarged view of FIG. 6B.

FIG. 7A is a perspective view of a diaphragm set according to another embodiment of the present disclosure; FIG. 7B is a cross-sectional view of the diaphragm set of FIG. 7A along plane C-C′; FIG. 7C is a partially enlarged view of FIG. 7B.

FIG. 8A is a perspective view of a diaphragm set according to another embodiment of the present disclosure; FIG. 8B is a cross-sectional view of the diaphragm set of FIG. 8A along plane D-D′; FIG. 8C is a partially enlarged view of FIG. 8B.

FIG. 9A is a perspective view of a diaphragm set according to another embodiment of the present disclosure; FIG. 9B is a cross-sectional view of the diaphragm set of FIG. 9A along plane E-E′ showing the way of the diaphragm set assembled to a chest piece; FIG. 9C is a partially enlarged view of FIG. 9B.

FIG. 10A is a perspective view of a capsule dispenser according to another embodiment of the present disclosure; FIG. 10B is a perspective view of the capsule dispenser of FIG. 10A when a peelable portion of the capsule dispenser is removed.

FIG. 11 is a flow chart of a method for replacing a diaphragm set of a stethoscope according to another embodiment of the present disclosure.

FIGS. 12A to 12D are schematic illustrations showing the steps of the method of FIG. 11.

### DETAILED DESCRIPTION OF THE PREFERRED EMBODIMENTS

The present disclosure will now be described more fully hereinafter with reference to the accompanying drawings, in which embodiments of the disclosure are shown. This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the term “and/or” includes any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

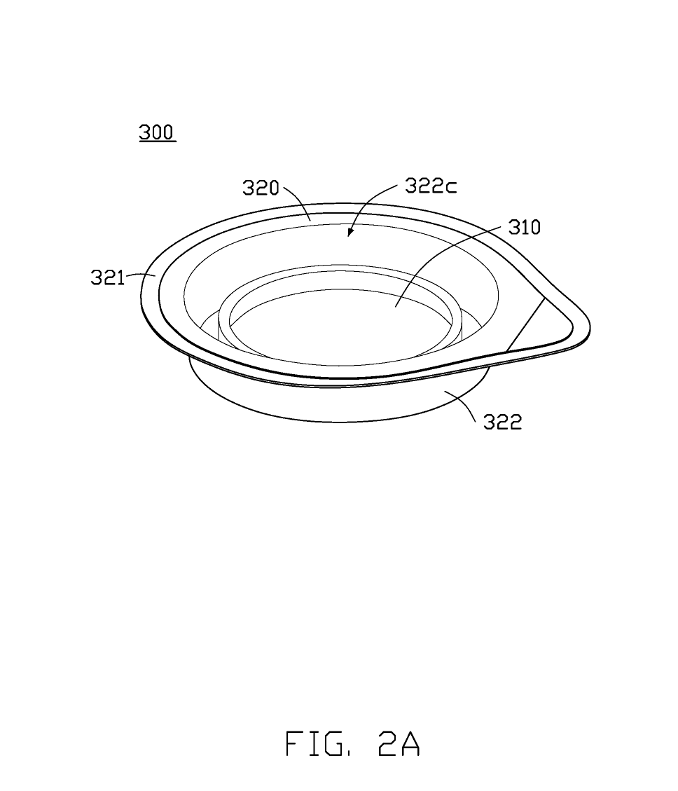

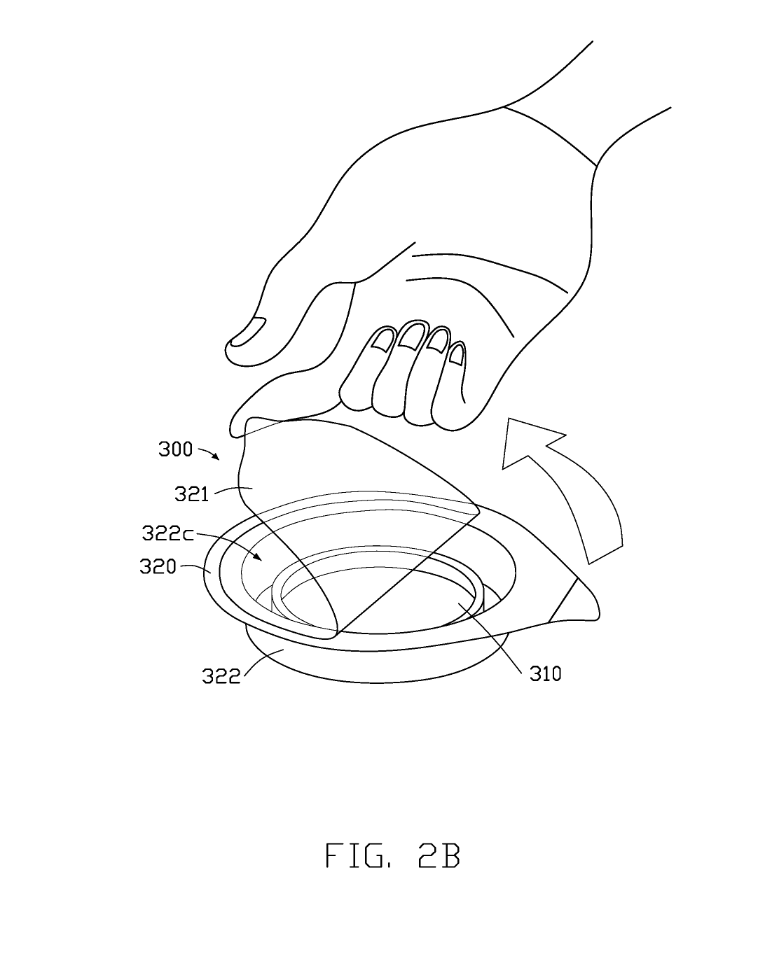

The description will be made as to the embodiments of the present disclosure in conjunction with the accompanying drawings in FIGS. 2A to 12D. Reference will be made to the drawing figures to describe the present disclosure in detail, wherein depicted elements are not necessarily shown to scale and wherein like or similar elements are designated by same or similar reference numeral through the several views and same or similar terminology.

The present disclosure will be further described hereafter in combination with figures.

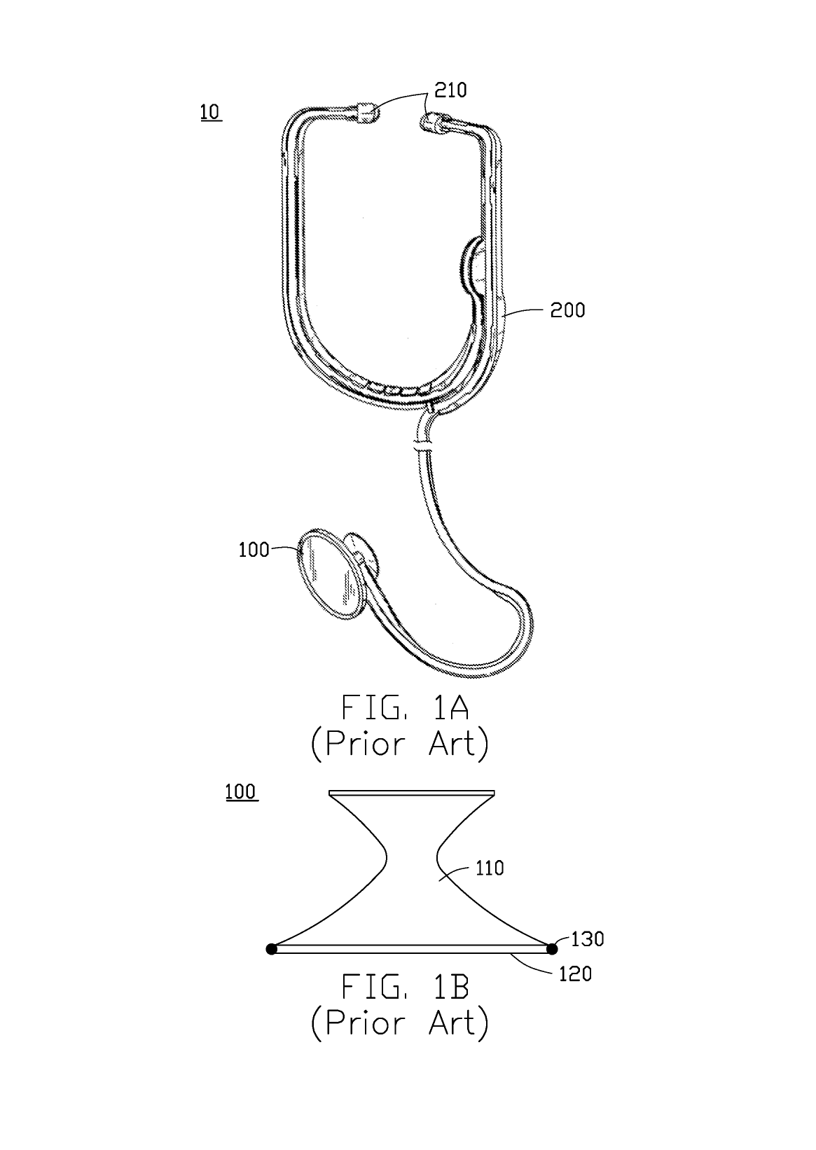

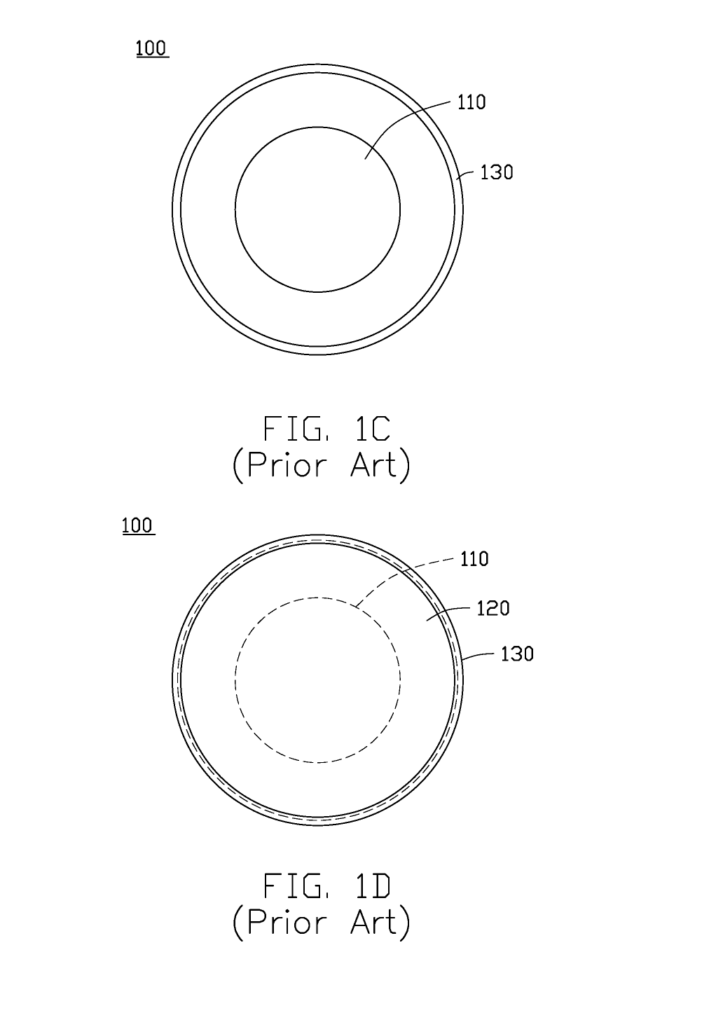

Referring to FIGS. 2A to 2C, perspective views of a disposable capsule containing a diaphragm set for a stethoscope according to an embodiment of the present disclosure are illustrated. As shown in FIGS. 2A to 2C, the disposable capsule 300 includes a diaphragm set 310 for a stethoscope 20 and a case 320 for receiving the diaphragm set 310. The stethoscope 20 usually includes a chest piece 210 and a stethoscope tube 220. The chest piece 210 and the stethoscope tube 220 of the stethoscope 20 can be referred to the chest piece 110 and the stethoscope tube 200 shown in FIGS. 1A and 1B without further description herein. In FIG. 2C, the stethoscope tube 220 is partially shown for brevity. As shown in FIG. 2A. The case 320 includes a holder 322 and a sealing film 321. The holder 322 has a capsule cavity 322c for receiving the diaphragm set 310. The sealing film 321 removably seals the capsule cavity 322c of the holder 322. As shown in FIG. 2B, when the sealing film 321 is peeled off or removed from the holder 322, the diaphragm set 310 is exposed and removable from the capsule cavity 322c of the holder 322. The diaphragm set 310 can be assembled to the chest piece 210 of the stethoscope 20 in or outside of the capsule cavity 322c, as shown in FIG. 2C. In one embodiment, one case 320 contains one diaphragm set 310. In one embodiment, the disposable capsule 300 may be made of various appropriate materials, such as foils, plastic, paper, laminates, etc. The disposable capsule 300 may have a round shape and may be sized with a diameter slightly greater than a diameter of the diaphragm set 310. The capsule cavity 322c may have an appropriate space for receiving the diaphragm set 310.

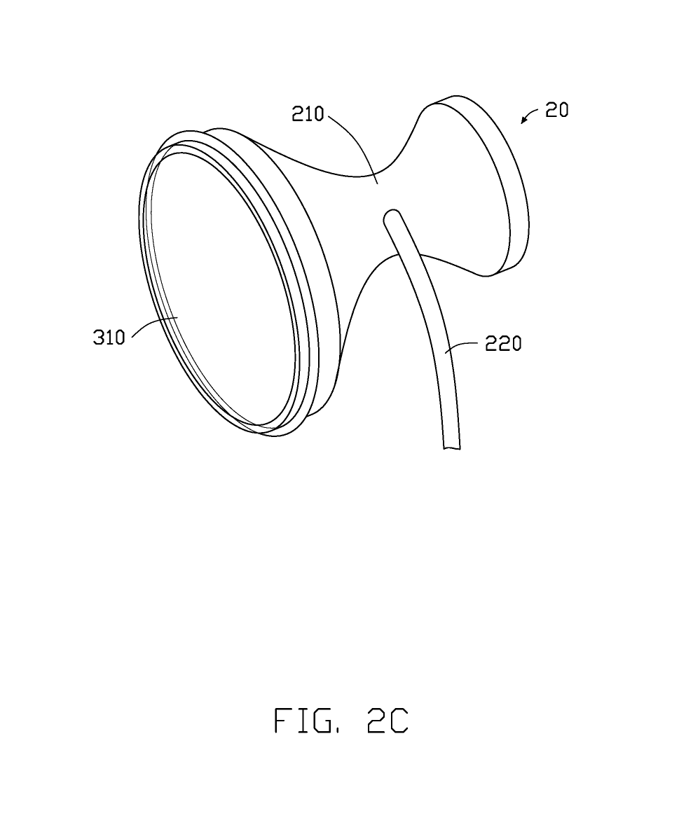

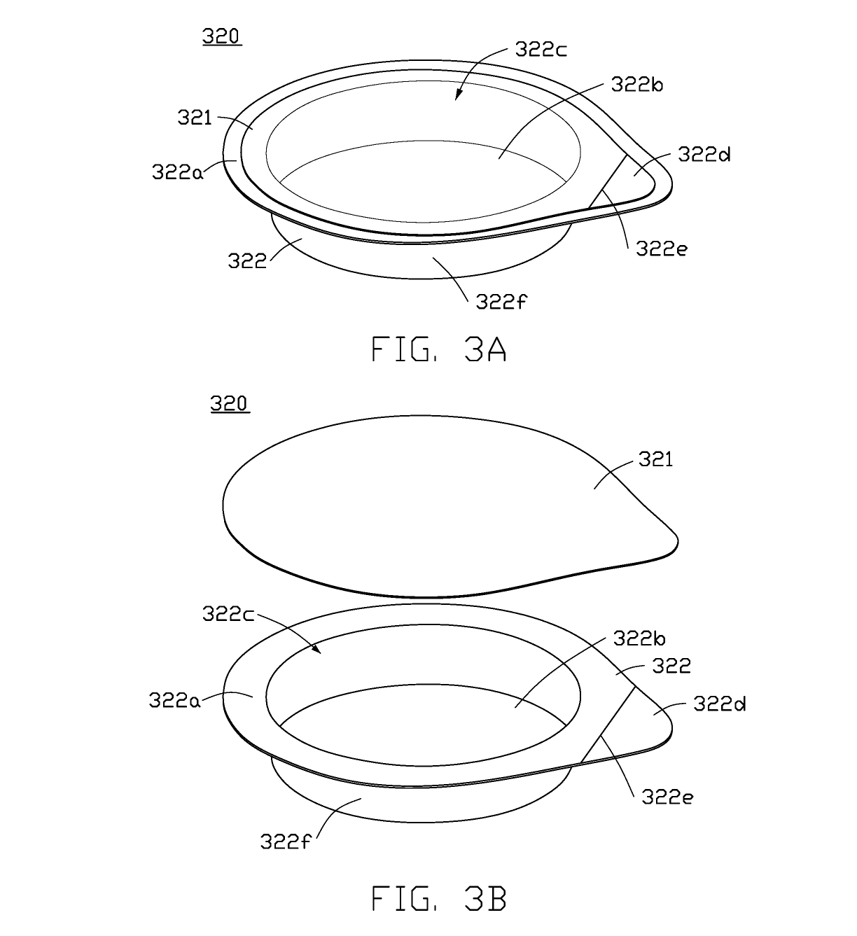

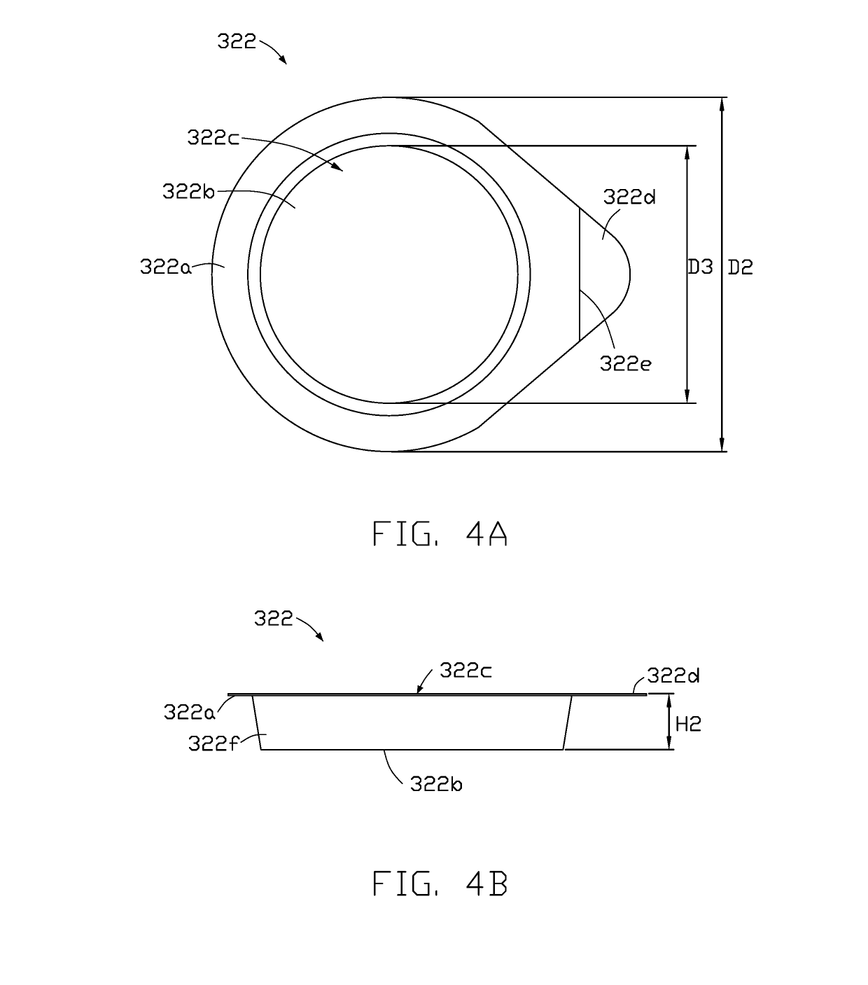

Referring to FIGS. 3A and 3B, a perspective view and an exploded view of the case 320 of FIG. 2A are illustrated. Referring to FIGS. 4A and 4B, a top view and a side view of the holder 322 of the case 320 of FIG. 3A are illustrated. The holder 322 includes a bottom portion 322b, a side portion 322f connected to the bottom portion 322b, and a flange portion 322a connected to an edge of the side portion 322f. The holder 322 can be made of a single piece of a thin film. A longitudinal direction of the flange portion 322a is parallel to a longitudinal direction of the bottom portion 322b. The bottom portion 322b and the side portion 322f form the capsule cavity 322c. The diaphragm set 310 is disposed in the capsule cavity 322c of the holder 322. The sealing film 321 seals the capsule cavity 322c by attaching to the flange portion 322a of the holder 322. The sealing film 321 is attached to the flange portion 322a of the holder 322 by hot-press gluing or ultrasonic welding. The flange portion 322a has an extended portion 322d and a groove 322e. The groove 322e is formed on the flange portion 322a by die-punching. The extended portion 322d is bendable along a longitudinal direction of the groove 322e. Removal of the sealing film 321 from the flange portion 322a is facilitated by bending and breaking the extended portion 322d along the longitudinal direction of the groove 322e. The holder 322 can be made of polystyrene (PS), polyethylene terephthalate (PET), polycarbonate (PC), metal, cardboard, or a combination thereof; but the embodiment is not limited thereto. The sealing film 321 can be made of paper, polystyrene (PS), polyethylene terephthalate (PET), polycarbonate (PC), or a combination thereof, such as “Easy-peel film” or “Tavik® paper”; but the embodiment is not limited thereto. The capsule cavity 322c has a diameter D3 within a range of 50 mm to 55 mm. The holder has a diameter D2 with a range of 65 mm to 80 mm and a height H2 of 10 mm to 15 mm.

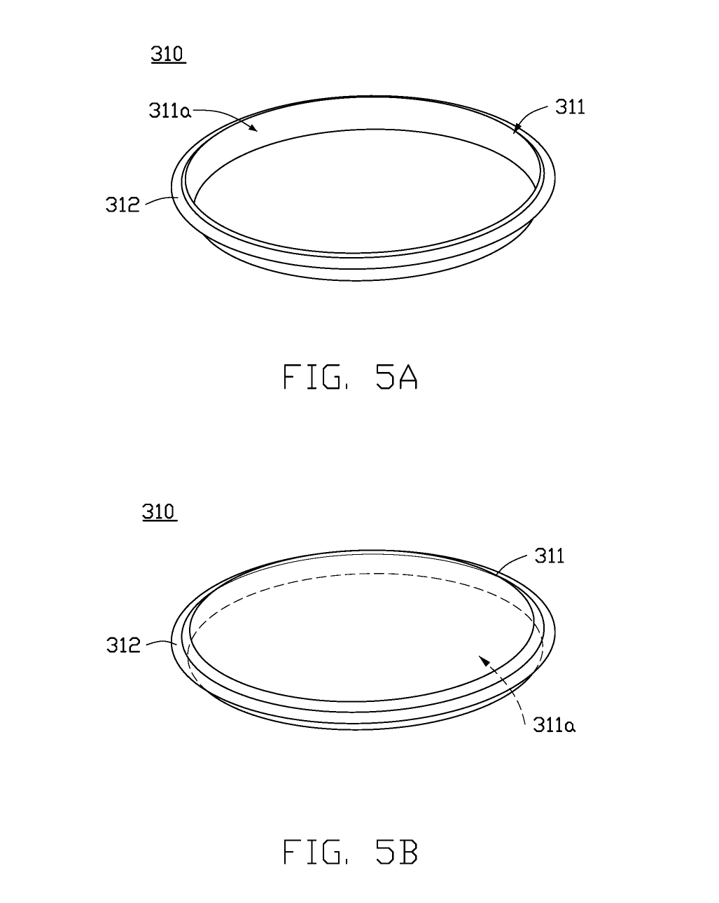

Referring to FIGS. 5A to 5E, perspective views, a top view, a bottom view, and a side view of the diaphragm set 310 of FIG. 2A are illustrated. Referring to FIG. 5F, a cross-sectional view of the diaphragm set of FIG. 5C along a plane A-A′ is illustrated. Referring to FIGS. 5G and 5H, partially enlarged views of FIG. 5F are illustrated. The diaphragm set 310 is configured to be assembled to the stethoscope 20. As shown in FIGS. 5A to 5G, the diaphragm set 310 includes a diaphragm 311 and an O-ring 312. The diaphragm 311 includes a bottom structure 311b and a side structure 311c connected to an edge of the bottom structure 311b. The side structure 311c and the bottom structure 311b are made of a single piece of a thin film. The side structure 311c is curved. In this embodiment, the side structure 311c is curved inwardly respect to a center line L1 of the diaphragm 311. The bottom structure 311b and the side structure 311c form a diaphragm cavity 311a for receiving the chest piece 210 of the stethoscope 20. The O-ring 312 is disposed around an outer surface 311d of the side structure 311c of the diaphragm 311. The outer surface 311d of the side structure 311c is curved and matches with a surface 312a of the O-ring 312, as shown in FIG. 5G. The side structure 311c may extend along the surface 312a of the O-ring 312 and partially cover the surface 312a of the O-ring 312. The diaphragm 311 has a diameter D1 within a range of 30 mm to 50 mm and a height H1 within a range of 1 mm to 3 mm. When the diaphragm set 310 is assembled to the chest piece 210 of the stethoscope 20, the curved side structure 311c allows the diaphragm 311 to easily fit onto an edge of the chest piece 210, and reduces the possibility of damaging the diaphragm 311 during the assembling process. The O-ring 312 may be made of silicon or other flexible materials. The chest piece 210 may include a groove (not shown in the figures) around the edge of the chest piece 210. When the diaphragm set 300 is assembled to the stethoscope 20, the O-ring 312 matches a shape of the groove of the chest piece 210 to allow the diaphragm 311 to be fixed onto the edge of the chest piece 210 of the stethoscope 20.

The diaphragm 311 is formed by stamping of a single piece of a thin film. As shown in FIG. 5H, the thin film of the diaphragm 311 includes a vibration layer 332 and a coating layer 331 coated on the vibration layer. The vibration layer 332 is disposed on a bottom surface of the bottom structure 311b of the diaphragm 311. The coating layer 331 is disposed on a top surface of the bottom structure 311b of the diaphragm 311. The coating layer 331 is made of silicon, soft touch paint, thermoplastic polyurethane (TPU), thermoplastic rubber (TPR), or a combination thereof. When the diaphragm set 310 is assembled to the chest piece 210 of the stethoscope 20, the coating layer 331 is in contact with the chest piece 210 of the stethoscope 20. In an embodiment, the coating layer 331 has a thickness within a range of 0.04 mm to 0.1 mm. In a preferred embodiment, the thickness of the coating layer 331 is within a range of 0.04 mm to 0.08 mm. The vibration layer 332 is made of polycarbonate, polyethylene terephthalate, glass fiber reinforced epoxy resin, or metal; but the embodiment is not limited thereto. The vibration layer 332 transmits a vibration generated by a patient's internal body sounds (such as heartbeats) to the chest piece 210 of the stethoscope 20. In an embodiment, the vibration layer 332 has a thickness within a range of 0.15 mm to 0.35 mm. In a preferred embodiment, the thickness of the vibration layer 332 is within a range of 0.15 mm to 0.22 mm. In a preferred embodiment, the coating layer 331 is formed on the vibration layer 331 by spray-coating and curing. The curing process is performed at a temperature of 45° C. to 50° C. The coating layer 331 has a higher elasticity than that of the vibration layer 332. The coating layer 331 provides good airtight sealing between the chest piece 310 and the diaphragm 311 of the diaphragm set 310, and therefore enhances the sensibility of the stethoscope 20.

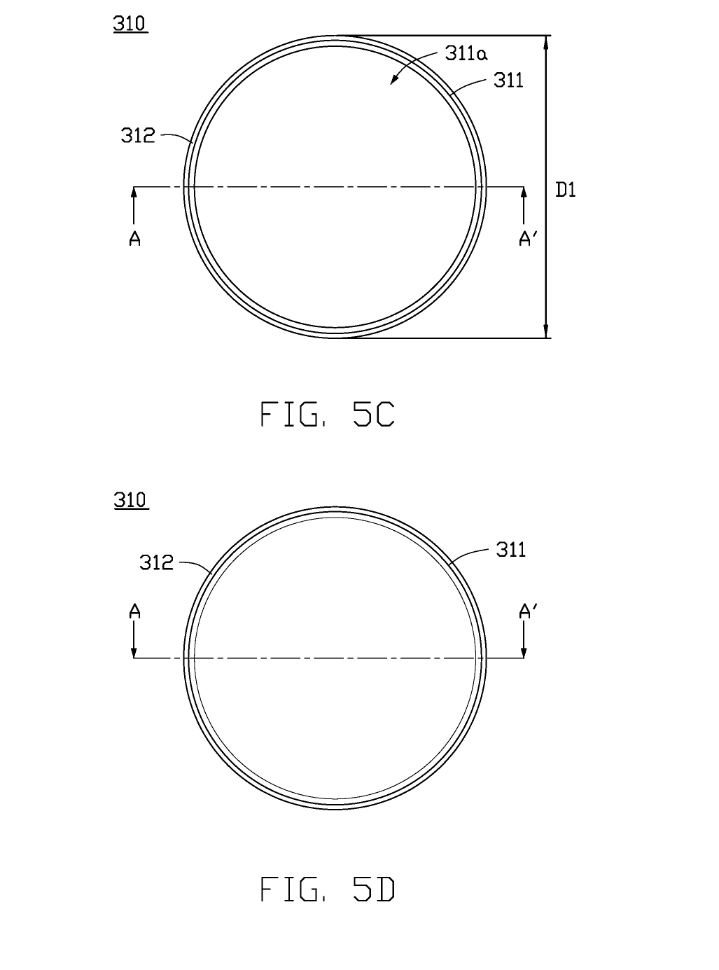

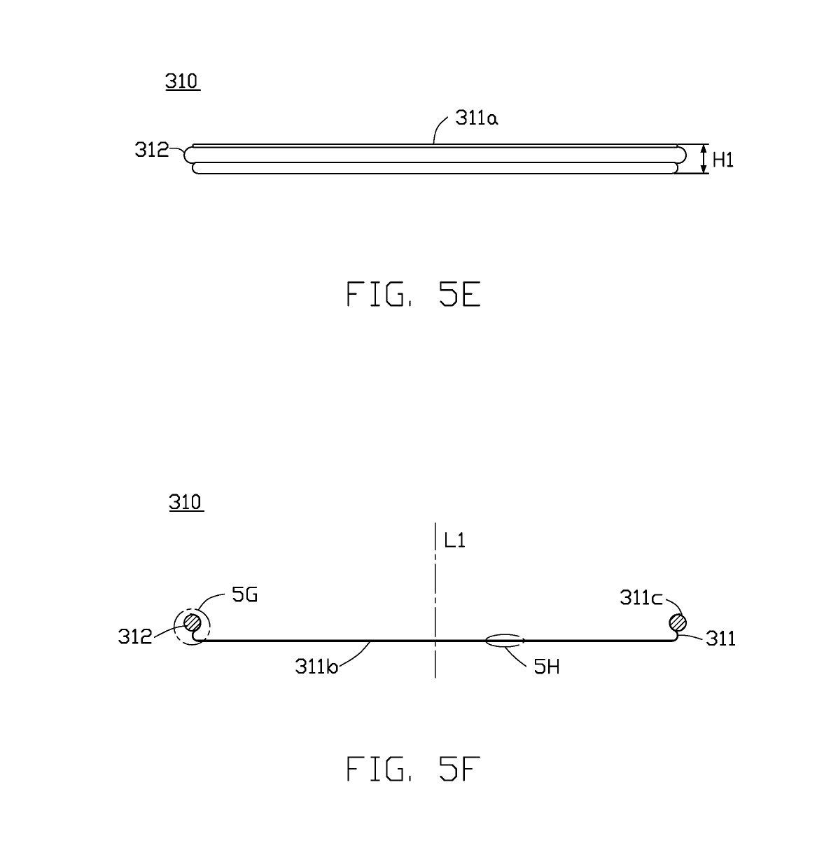

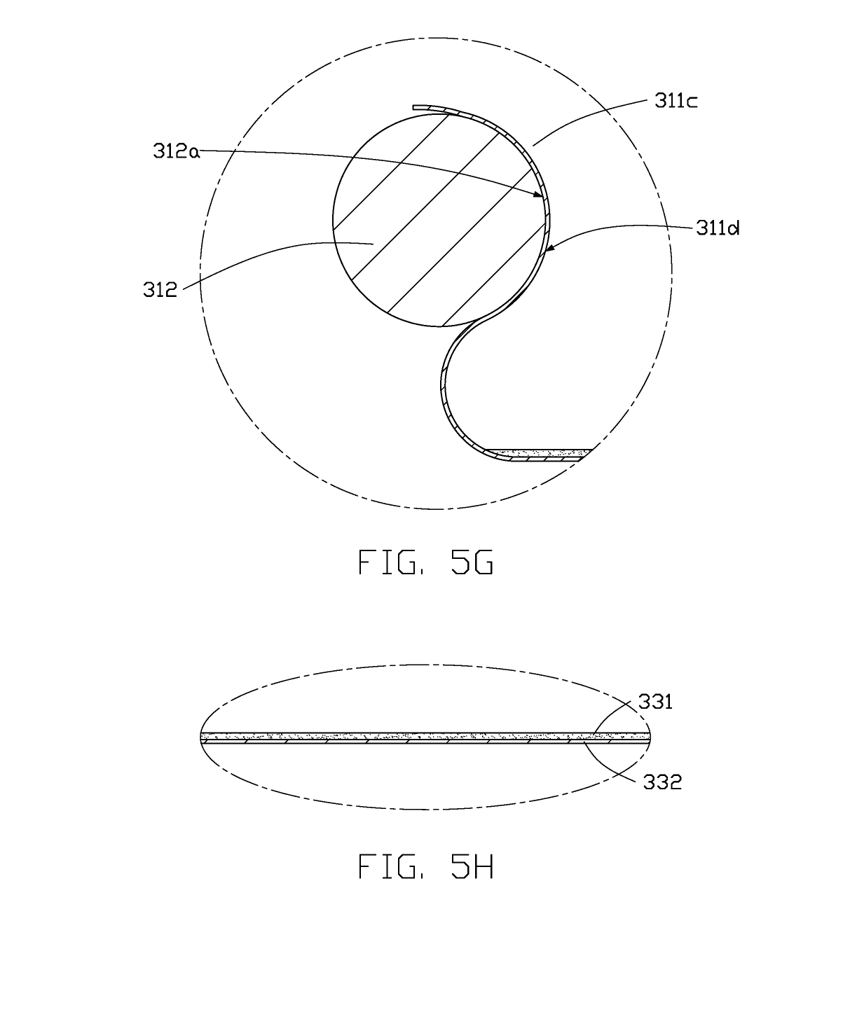

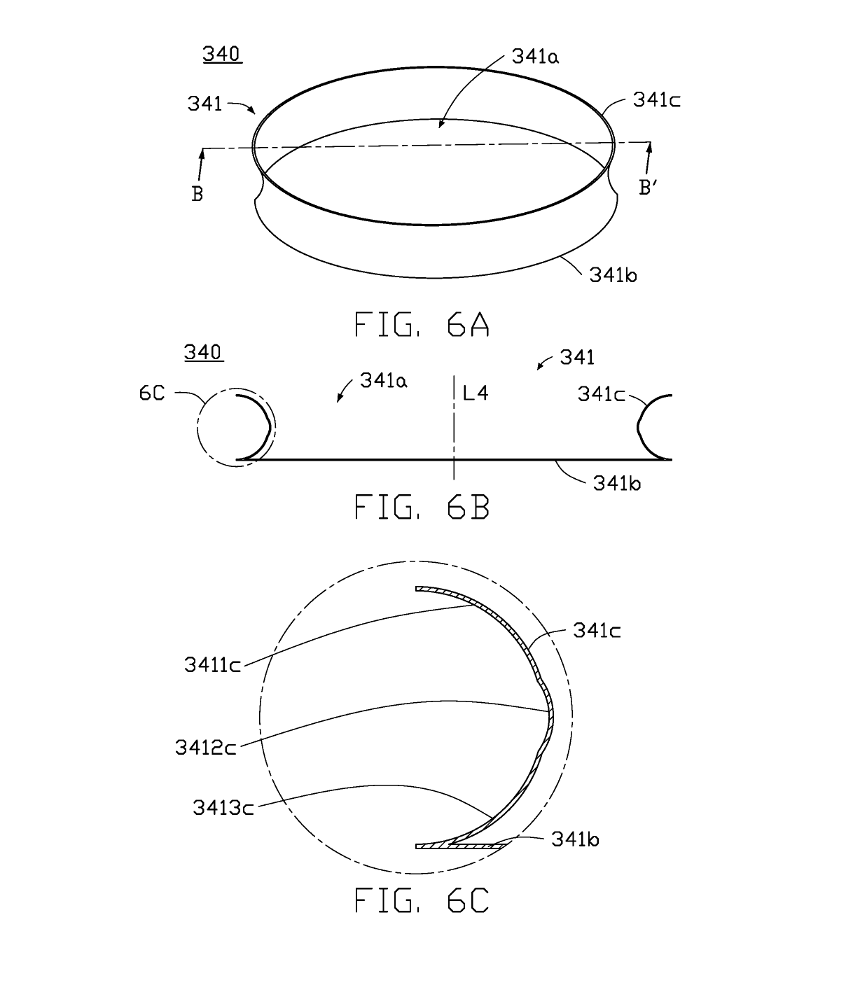

Referring to FIG. 6A, a perspective view of the diaphragm set according to another embodiment of the present disclosure is illustrated. Referring to FIGS. 6B and 6C, a cross-sectional view and a partially enlarged view of the diaphragm set of FIG. 6A are illustrated. In this embodiment, the diaphragm set 340 for a stethoscope having a chest piece includes a diaphragm 341. The diaphragm 341 includes a bottom structure 341b and a side structure 341c connected to an edge of the bottom structure 341b. The side structure 341c and the bottom structure 341b are made of a single piece of a thin film. The side structure 341c is curved. The bottom structure 341b and the side structure 341c form a diaphragm cavity 341a for receiving the chest piece of the stethoscope. As shown in FIG. 6B, the side structure 341c is curved inwardly with respect to a center line L4 of the diaphragm 341. As shown in FIG. 6C, the side structure 341c includes a connecting portion 3413c connected to the bottom structure 341b, a middle portion 3412c connected to the connecting portion 3413c, and an upper portion 3411c connected to the middle portion 3412c. A radius of curvature of the middle portion 3412c is smaller than radius of curvatures of the connecting portion 3413c and the upper portion 3411c. The diaphragm set 340 may further includes an O-ring disposed around an outer surface of the side structure 341c of the diaphragm 341. The O-ring can be referred to the O-ring 312 shown in FIGS. 5A to 5F without further description. Other features of the diaphragm 341 (such as materials or sizes) can be referred to the diaphragm 311 described in the previous embodiment.

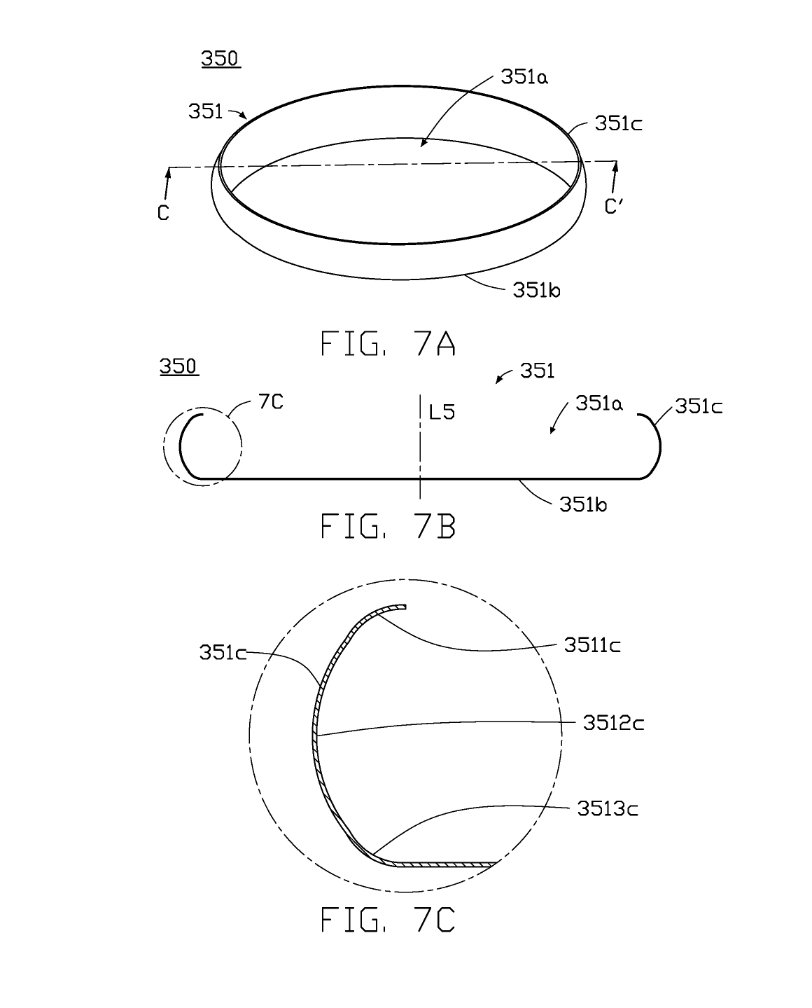

Referring to FIG. 7A, a perspective view of the diaphragm set according to another embodiment of the present disclosure is illustrated. Referring to FIGS. 7B and 7C, a cross-sectional view and a partially enlarged view of the diaphragm set of FIG. 7A are illustrated. In this embodiment, the diaphragm set 350 for a stethoscope having a chest piece includes a diaphragm 351. The diaphragm 351 includes a bottom structure 351b and a side structure 351c connected to an edge of the bottom structure 351b. The side structure 351c and the bottom structure 351b are made of a single piece of a thin film. The side structure 351c is curved. The bottom structure 351b and the side structure 351c form a diaphragm cavity 351a for receiving the chest piece of the stethoscope. As shown in FIG. 7B, the side structure 351c is curved outwardly with respect to a center line L5 of the diaphragm 351. As shown in FIG. 7C, the side structure 351c includes a connecting portion 3513c connected to the bottom structure 351b, a middle portion 3512c connected to the connecting portion 3513c, and an upper portion 3511c connected to the middle portion 3512c. A radius of curvature of the middle portion 3512c is greater than radius of curvatures of the connecting portion 3513c and the upper portion 3511c. Other features of the diaphragm 351 (such as materials or sizes) can be referred to the diaphragm 311 described in the previous embodiment.

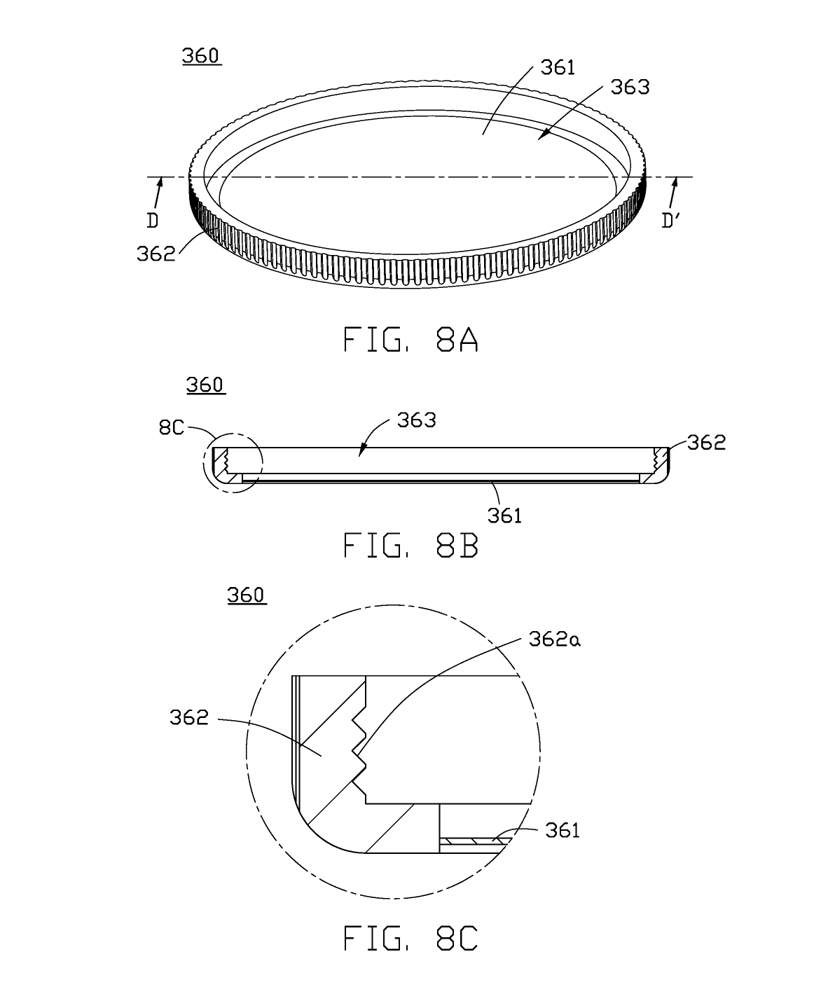

Referring to FIG. 8A, a perspective view of the diaphragm set according to another embodiment of the present disclosure is illustrated. Referring to FIGS. 8B and 8C, a cross-sectional view and a partially enlarged view of the diaphragm set of FIG. 8A are illustrated. In this embodiment, the diaphragm set 360 for a stethoscope having a chest piece includes a diaphragm 361 and a rim element 362. In this embodiment, the diaphragm 361 is a flat circular membrane. The rim element 362 couples to an edge of the diaphragm 361 to form a cavity 363 for receiving the chest piece of the stethoscope. The rim element 362 may be made of aluminum alloy. When the rim element 362 is made by aluminum alloy, as shown in FIG. 8C, the rim element 362 may have at least one screw thread 362a formed around an inner surface of the rim element 362. When the diaphragm set 360 is assembled to the chest piece of the stethoscope, the diaphragm set 360 can be screwed to the chest piece by matching grooves formed on an outer surface of the edge of the chest piece. Alternatively, the rim element 362 may have at least one groove formed around an inner surface of the rim element 362 for matching screw threads formed on the outer surface of the edge of the chest piece. In this embodiment, the rim element 362 has an L-shaped cross-section.

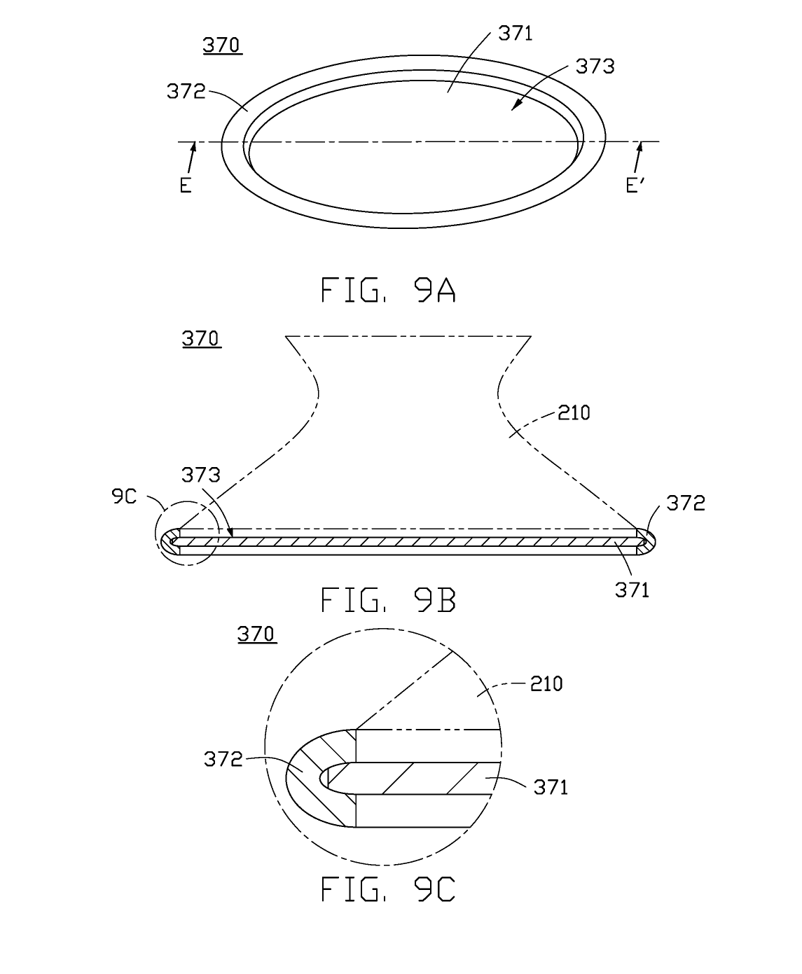

Referring to FIG. 9A, a perspective view of the diaphragm set according to another embodiment of the present disclosure is illustrated. Referring to FIGS. 9B and 9C, a cross-sectional view and a partially enlarged view showing the way the diaphragm set of FIG. 9A assembled to a chest piece 210 are illustrated. In this embodiment, the diaphragm set 370 for a stethoscope having a chest piece 210 includes a diaphragm 371 and a rim element 372. In this embodiment, the diaphragm 371 is a flat circular membrane. The rim element 372 couples to an edge of the diaphragm 371 to form a cavity 373 for receiving the chest piece of the stethoscope. The rim element 372 may be made of silicon or rubber. As shown in FIG. 9C, the rim element 372 has a C-shaped cross-section.

Referring to FIGS. 10A and 10B, a capsule dispenser for the disposable capsule 300 according to another embodiment of the present disclosure is illustrated. The capsule dispenser 400 includes a dispenser housing 410 and at least one disposable capsule 300 disposed in the dispenser housing 410. The disposable capsule 300 is described in the aforementioned embodiments; no further description is to be repeated herein. Preferably, one capsule dispenser 400 is capable of receiving 10 to 20 disposable capsules 300. The disposable capsules 300 are stacked in an inner space of the dispenser housing 410. The dispenser housing 410 has a peelable portion 411. The peelable portion 411 is configured to be peeled off from the dispenser housing 410 to form a dispensing hole 413 on one end of the dispenser housing 410 to allow the disposable capsules 300 to be removed from the dispenser housing 410. The dispenser housing 410 may further include a fixing member 412 to allow the dispenser housing 410 to be fixed at a desired place, such as a wall or a table. The fixing member 412 can be designed in the way of hooking or pasting. The capsule dispenser 400 allows the disposable capsules 300 to be neatly stored and easily retrieved.

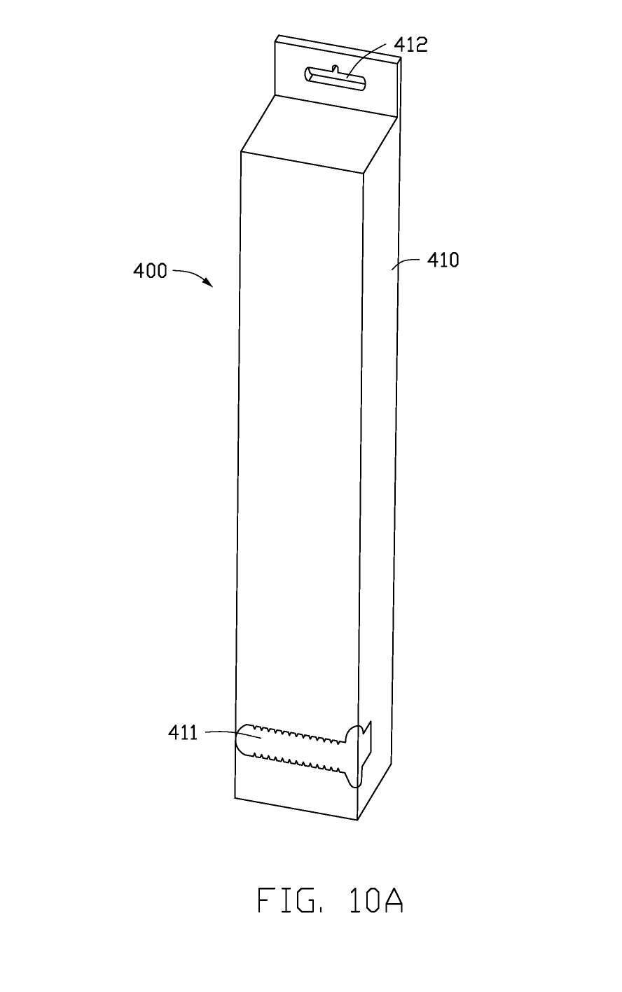

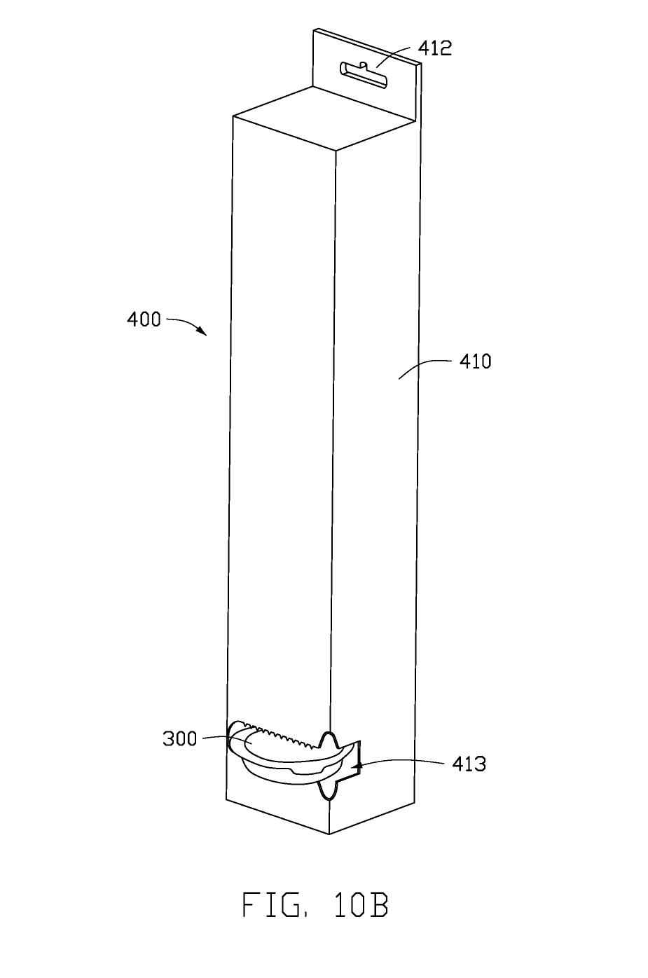

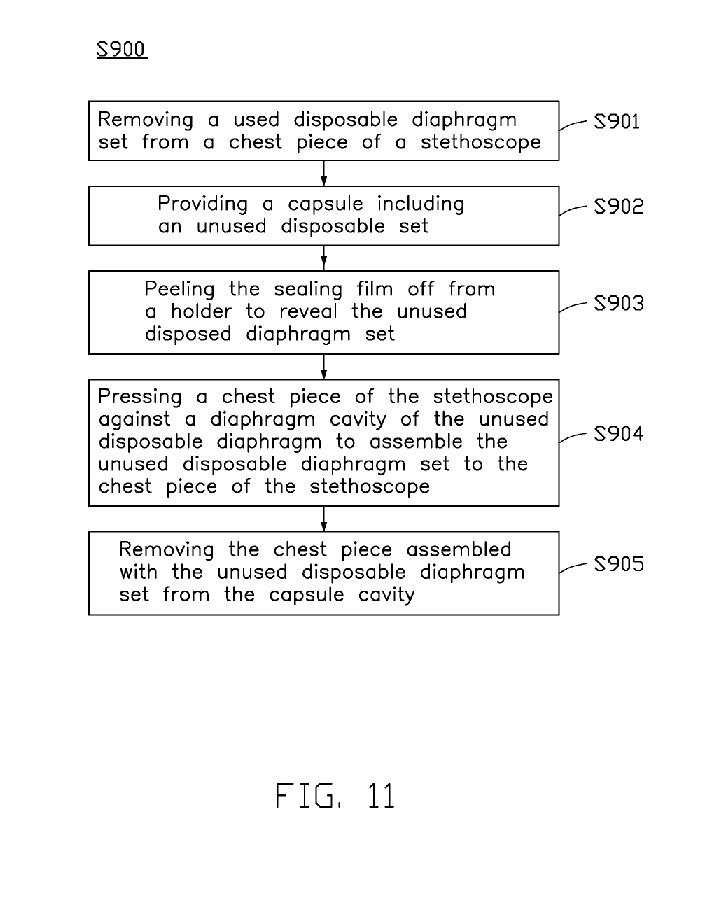

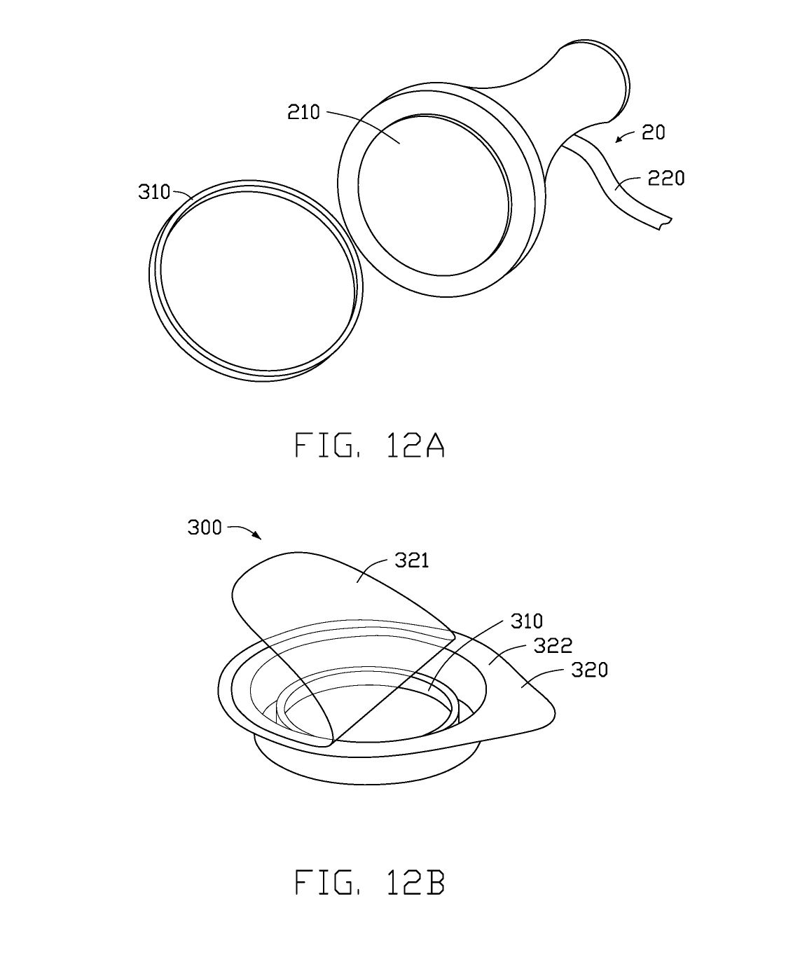

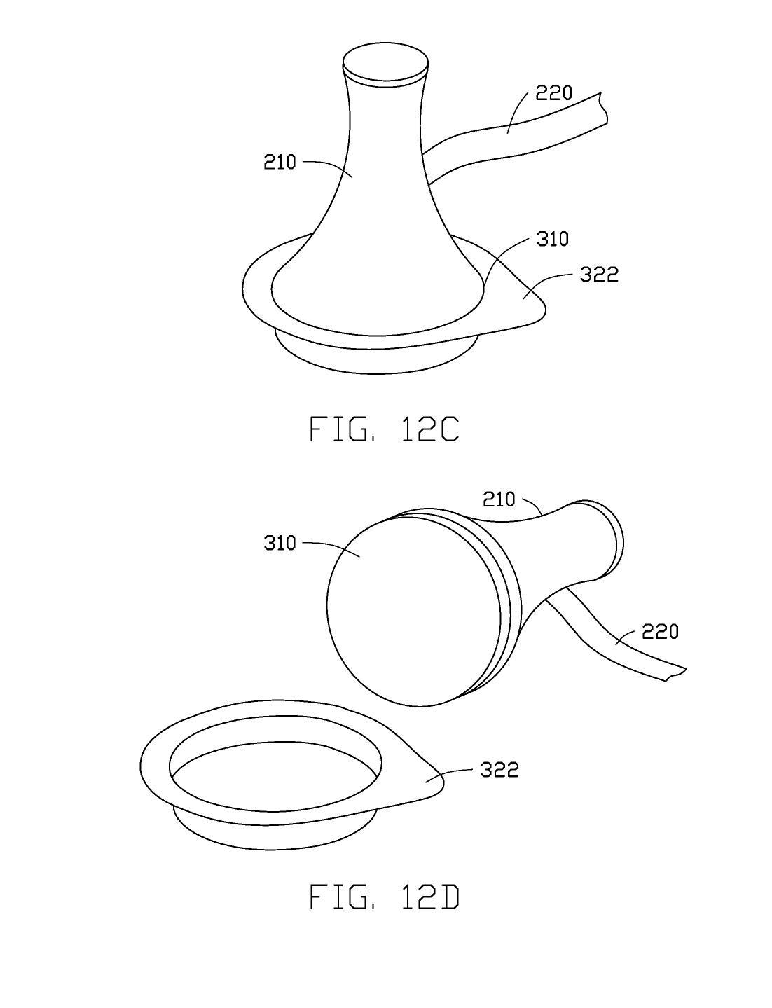

Referring to FIG. 11, a flow chart of a method for replacing a diaphragm set for a stethoscope according to another embodiment of the present disclosure is illustrated. The stethoscope and the diaphragm set can be referred to the stethoscope and the diaphragm set of the aforementioned embodiments. Referring to FIGS. 12A to 12D, schematic illustrations showing the steps of the method of FIG. 11 are provided. In the embodiment, the stethoscope includes a chest piece and a stethoscope tube connected to the chest piece. According to an embodiment, the method S900 for replacing a diaphragm set for a stethoscope includes steps S901 to S905.

Step S901: Removing a used diaphragm set from the chest piece of the stethoscope, as shown in FIG. 12A.

Step S902: Providing a disposable capsule 300 having an unused diaphragm set 310 and a case 320 for receiving the unused diaphragm set. The diaphragm of the diaphragm set 310 includes a bottom structure and a side structure connected to an edge of the bottom structure. The bottom structure and the side structure form a diaphragm cavity for receiving the chest piece of the stethoscope. The O-ring is disposed around an outer surface of the side structure of the diaphragm. The outer surface of the side structure is curved and matches with a surface of the O-ring. The case 320 includes a holder having a capsule cavity for receiving the unused diaphragm set and a sealing film removably sealing the cavity of the holder. The disposable capsule can be referred to the disposable capsule 300 of the aforementioned embodiments; no further description is to be repeated herein. The disposable capsule can be obtained from the capsule dispenser 400 as shown in FIGS. 10A and 10B.

Step S903: Peeling the sealing film off from the holder to expose the unused disposed diaphragm set in the capsule cavity, as shown in FIG. 12B.

Step S904: Pressing the chest piece of the stethoscope against the diaphragm cavity of the diaphragm to assemble the unused diaphragm set to the chest piece of the stethoscope, as shown in FIG. 12C.

Step S905: Removing the chest piece assembled with the unused diaphragm set from the capsule cavity, as shown in FIG. 12D.

The method S900 of the embodiment of the present disclosure allows users to replace a used diaphragm set with an unused diaphragm set from the disposable capsule without touching the unused diaphragm set. Therefore, the method S900 can prevent the contamination and damage of the unused diaphragm set.

As described above, the diaphragm set of the embodiments of the present disclosure allows the user to assemble the diaphragm set to a chest piece of a stethoscope without damaging the diaphragm. In addition, the disposable capsule containing the diaphragm set of the embodiments of the present disclosure allows the user to easily replace the diaphragm set of the stethoscope without touching the diaphragm set, therefore preventing contamination and damage of the diaphragm. Furthermore, the capsule dispenser allows the disposable capsules to be neatly stored and easily retrieved.

The embodiments shown and described above are only examples. Many details are often found in the art such as the other features of a diaphragm set and a disposable capsule thereof. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A diaphragm set for a stethoscope having a chest piece, comprising:
a diaphragm comprising a bottom structure and a side structure connected to an edge of the bottom structure, wherein the bottom structure and the side structure of the diaphragm form a diaphragm cavity for receiving the chest piece of the stethoscope.

2. The diaphragm set of claim 1, wherein the side structure is curved inwardly with respect to a center line of the diaphragm.

3. The diaphragm set of claim 2, wherein the side structure comprises:
a connecting portion connected to the bottom structure;
a middle portion connected to the connecting portion; and
an upper portion connected to the middle portion;
wherein a radius of curvature of the middle portion is smaller than radius of curvatures of the connecting portion and the upper portion.

4. The diaphragm set of claim 2, further comprising an O-ring disposed around an outer surface of the side structure of the diaphragm.

5. The diaphragm set of claim 1, wherein the side structure is curved outwardly with respect to a center line of the diaphragm.

6. The diaphragm set of claim 5, wherein the side structure comprises:
a connecting portion connected to the bottom structure;
a middle portion connected to the connecting portion; and
an upper portion connected to the middle portion;
wherein a radius of curvature of the middle portion is greater than radius of curvatures of the connecting portion and the upper portion.

7. The diaphragm set of claim 1, wherein the side structure and the bottom structure are made of a single piece of thin film, the thin film comprises a vibration layer and a coating layer coated on the vibration layer, when the diaphragm set is assembled to the chest piece of the stethoscope, the coating layer is in contact with the chest piece of the stethoscope.

8. A disposable capsule comprising:
a case comprising:
a holder having a capsule cavity; and
a sealing film sealing the capsule cavity of the holder; and
a diaphragm set for a stethoscope having a chest piece disposed in the capsule cavity of the case.

9. The disposable capsule of claim 8, wherein the diaphragm set comprising a diaphragm and a rim element, the rim element couples to an edge of the diaphragm to form a cavity for receiving the chest piece of the stethoscope.

10. The disposable capsule of claim 8, wherein when the sealing film is peeled off from the holder, the diaphragm set is removable from the capsule cavity of the holder.

11. The disposable capsule of claim 8, wherein the holder comprises a bottom portion, a side portion connected to the bottom portion, and a flange portion connected to an edge of the side portion, the holder is made of a single piece of a thin film, a longitudinal direction of the flange portion is parallel to a longitudinal direction of the bottom portion, the bottom portion and the side portion form the capsule cavity, and the sealing film seals the capsule cavity by attaching to the flange portion of the holder.

12. The disposable capsule of claim 11, wherein the sealing film is attached to the flange portion of the holder by hot-press gluing or ultrasonic welding.

13. The disposable capsule of claim 11, wherein the flange portion has an extended portion and a groove, the extended portion is bendable along a longitudinal direction of the groove.

14. The disposable capsule of claim 13, wherein the groove is formed on the flange portion by die-punching.

15. The disposable capsule of claim 8, wherein the holder is made of polystyrene, polyethylene terephthalate, polycarbonate, metal, cardboard, or a combination thereof.

16. The disposable capsule of claim 8, wherein the sealing film is made of paper, polystyrene, polyethylene terephthalate, polycarbonate, or a combination thereof.

17. The disposable capsule of claim 8, wherein the diaphragm set comprises:
a diaphragm comprising a bottom structure and a side structure connected to an edge of the bottom structure, wherein the bottom structure and the side structure form a diaphragm cavity for receiving the chest piece of the stethoscope.

18. The disposable capsule of claim 17, wherein the side structure of the diaphragm is curved inwardly with respect to a center line of the diaphragm.

19. The disposable capsule of claim 18, wherein the side structure of the diaphragm comprises:
a connecting portion connected to the bottom structure;
a middle portion connected to the connecting portion; and
an upper portion connected to the middle portion;
wherein a radius of curvature of the middle portion is smaller than radius of curvatures of the connecting portion and the upper portion.

20. The disposable capsule of claim 18, wherein the diaphragm set further comprises an O-ring disposed around an outer surface of the curved side structure of the diaphragm.

21. The disposable capsule of claim 17, wherein the side structure of the diaphragm is curved outwardly with respect to a center line of the diaphragm.

22. The disposable capsule of claim 21, wherein the side structure of the diaphragm comprises:
a connecting portion connected to the bottom structure;
a middle portion connected to the connecting portion; and
an upper portion connected to the middle portion;
wherein a radius of curvature of the middle portion is greater than radius of curvatures of the connecting portion and the upper portion.

23. The disposable capsule of claim 17, wherein the diaphragm has a diameter within a range of 30 mm to 50 mm and a height within a range of 1 mm to 3 mm.

24. The disposable capsule of claim 17, wherein the capsule cavity has a diameter within a range of 50 mm to 55 mm, the holder has a diameter with a range of 65 mm to 80 mm and a height of 10 mm to 15 mm.

25. A capsule dispenser comprising:
a dispenser housing having a peelable portion; and
at least one disposable capsule disposed in the dispenser housing, comprising:
a diaphragm set for a stethoscope having a chest piece, comprising:
a diaphragm comprising a bottom structure and a side structure connected to an edge of the bottom structure, wherein the bottom structure and the side structure of the diaphragm form a diaphragm cavity for receiving the chest piece of the stethoscope; and
a case for receiving the diaphragm set comprising:
a holder having a capsule cavity for receiving the diaphragm set; and
a sealing film sealing the capsule cavity of the holder;
wherein the peelable portion is configured to be peeled off from the dispenser housing to form a dispensing hole on the dispenser housing to allow the disposable capsule to be removed from the dispenser housing.
